(19) United States
(12) Patent Application Publication
(10) Pub. No.: US 2007/0299155 A1
(43) Pub. Date: Dec. 27, 2007

(54) Biodegradable polymer adhesion barriers

(75) Inventors: Kenneth W. Carpenter, San Diego, CA (US); Huashi Zhang, San Diego, CA (US)

Correspondence Address: DLA PIPER US LLP, 4365 EXECUTIVE DRIVE, SUITE 1100, SAN DIEGO, CA, 92121-2133, US

(73) Assignee: MediVas, LLC, San Diego, CA (US)

(21) Appl. No.: 11/811,026

(22) Filed: Jun. 8, 2007

Related U.S. Application Data
(60) Provisional application No. 60/812,472, filed on Jun. 9, 2006.
(60) Provisional application No. 60/840,290, filed on Aug. 24, 2006.

(51) Int. Cl. C08G 63/685; A61M 35/00

(52) U.S. Cl. 523/105; 524/379; 526/312; 528/369; 604/290

(57) ABSTRACT

The present invention provides adhesion barrier compositions based on a solution of at least one of a biodegradable polyester amide (PEA), polyester urethane (PEUR), or polyester urea (PEU) polymers, dissolved in a biocompatible solvent. The compositions can be applied to a tissue surface, such as in open surgery, as a viscous liquid which forms an adhesive film upon being sprayed or painted onto the tissue surface. Alternatively, the composition can be applied to the tissue surface as a preformed solid layer or double layer (either porous or non-porous) that adheres to the tissue surface. In open surgery, the invention adhesion barrier compositions are used to separate opposing tissue surfaces or tissue-organ surfaces while injured tissues heal, for example in the abdomen or pelvis

### RELATED APPLICATIONS

This application claims priority under 35 U.S.C. §119(e) of U.S. Provisional applications, Ser. Nos. 60/812,472 filed Jun. 9, 2006 and 60/840,290 filed Aug. 24, 2006 each of which is hereby incorporated by reference in its entirety

### FIELD OF THE INVENTION

The invention relates generally to polymer films and implants and in particular to sprayable and solid biodegradable polymer adhesion barriers for prevention of post surgical adhesions.

### BACKGROUND OF THE INVENTION

Adhesions are fibrous connections between tissues and organs that form as a response to tissue injury. Tissue injury is a natural consequence of such treatments as open abdominal, thoracic, and pelvic surgery, radiation to abdominal and pelvic areas, or other diseases that cause tissue injury to interior body sites, such as endometriosis. In particular, adhesions are very common following open abdominal and pelvic surgery. The type of surgery, as well as factors such as the length of surgery, associated illness, and other treatments, may influence the body's reaction to tissue injury.

Adhesion-related surgical complications include small bowel obstruction, infertility, and chronic pelvic pain. For example, adhesions can lead to infertility when an abnormal orientation of the ovary, fallopian tubes, or uterus is caused, thereby blocking the egg from traveling into the uterus. Adhesions from a previous procedure can also complicate a second surgery, whether the surgery is planned or unexpected. In addition, the abnormal orientation of tissues and organs caused by adhesions may lead to discomfort and chronic pain. A frequently used procedure for treating chronic pelvic pain is surgery to cut through any adhesions present in the abdomen or pelvis, for example, before performing an intended procedure.

Adhesion barriers work by separating opposing tissue surfaces or tissue-organ surfaces while injured tissues in the abdomen and pelvis heal. Ingrowth of scar tissue and the formation or reformation of adhesions immediately adjacent to the barrier film is thus prevented.

One type of known adhesion barrier is a thin film composed of chemically modified sugars, some of which occur naturally in the human body. The film adheres to tissues to which it is applied, and is slowly absorbed into the body over a period of about a week.

Another type of adhesion barrier is made of an amorphous bioresorbable copolymer, 70:30 Poly(L-lactide-co-D, L-lactide), which is designed to match the natural lactic acid produced in the body. As an inert material, the body accepts the polymer and processes it through the normal channels of bulk hydrolysis, followed by further breakdown in the liver into CO₂ and H₂O. Still another type of adhesion barrier based on Polyethyleneglycol (PEG) is applied as two liquids, which are simultaneously sprayed onto the target area to form a soft adherent hydrogel. Within about one week, the hydrogel undergoes hydrolysis and is cleared from the body by the kidneys.

Despite these advances in the art, the need exists for new and better bioabsorbable adhesion barrier compositions to be used for prevention of post surgical adhesions.

### A BRIEF DESCRIPTION OF THE FIGURES

FIG. 1 is a graph showing. GPC traces for macrophage degradation of PEA.Ac.Bz. Trace A—level of macrophage degradation on day 14, Trace B—level of macrophage degradation on day 10, Trace C—level of macrophage degradation on day 7, Trace D—level of macrophage degradation on day 3, Trace E—control media only, day 14, Trace F—undegraded PEA.Ac.Bz, no macrophages. ↑=starting material; ↑↑↑↑=degradation products.

FIG. 2 is a graph showing GPC traces for macrophage degradation of PEA.Ac.TEMPO. Trace A—level of macrophage degradation on day 14, Trace B—level of macrophage degradation on day 10, Trace C—level of macrophage degradation on day 7, Trace D—level of macrophage degradation on day 3, Trace E—control media only, day 14, Trace F—undegraded PEA.Ac.TEMPO, no macrophages. ↑=starting material; ↑↑↑↑=degradation products.

FIG. 3 is a graph showing the rate of phenotypic progression of monocytes-to-macrophages and contact-induced fusion to form multinucleated cells on PEA and other test polymers over three weeks of culture. 50:50 poly(D,L-lactide-co-glycolide)=PLGA, poly(n-butyl methacrylate)=PBMA, and tissue culture-treated polystyrene=TCPS.

FIG. 4 is a graph showing secretion of IL-1β by monocytes incubated on PEAs and indicated test polymers.

FIG. 5 is a graph showing secretion of IL-6 by monocytes incubated for 24 hours on PEAs, PLGA 34K and 73K and PBMA

FIG. 6 is a graph showing secretion of Interleukin-1 receptor antagonist, a naturally occurring inhibitor of IL-1β, by adherent monocytes incubated on PEAs and on indicated test polymers.

### SUMMARY OF THE INVENTION

In one embodiment, the invention provides an adhesion barrier composition in which at least one biodegradable adherent polymer is dissolved in a biocompatible liquid solvent, wherein the polymer contains at least one of a poly(ester amide) (PEA) having a chemical formula described by structural formula (I), 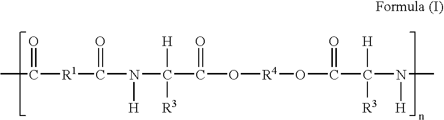
wherein n ranges from about 5 to about 150; R¹ is independently selected from the group consisting of (C₂-C₂₀) alkylene, (C₂-C₂₀) alkenylene, α,ω-bis(4-carboxyphenoxy) (C₁-C₈) alkane, residues of saturated and unsaturated adhesion preventing di-acids, residues of α,ω-alkylene dicarboxylates of formula (III), and combinations thereof; wherein R⁵ and R⁷ in Formula (III) are each independently selected from (C₂-C₁₂) alkylene or (C₂-C₁₂) alkenylene; the R³s in individual n monomers are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing di-acids, and combinations thereof;

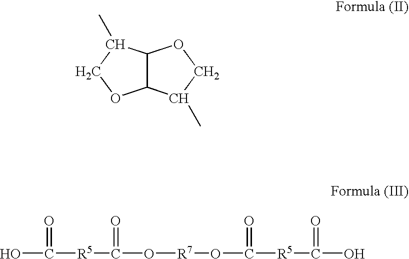

or a PEA having a chemical formula described by structural formula (IV), 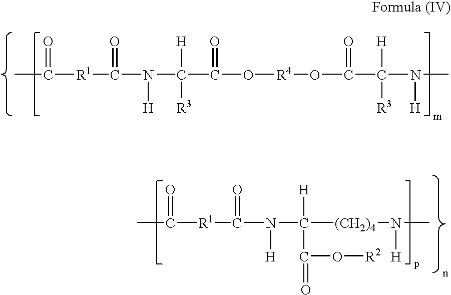
wherein n ranges from about 5 to about 150, m ranges about 0.1 to 0.9: p ranges from about 0.9 to 0.1; wherein R¹ is independently selected from the group consisting of (C₂-C₂₀) alkylene, (C₂-C₂₀) alkenylene, α,ω-bis(4-carboxyphenoxy) (C₁-C₈) alkane, and residues of saturated and unsaturated adhesion preventing di-acids, residues of α,ω-alkylene dicarboxylates of formula (III), and combinations thereof; wherein R⁵ and R⁷ in Formula (III) are each independently selected from (C₂-C₁₂) alkylene or (C₂-C₁₂) alkenylene; each R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R³s in individual m monomers are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols and combinations thereof;

or a poly(ester urethane) (PEUR) having a chemical formula described by structural formula (V), 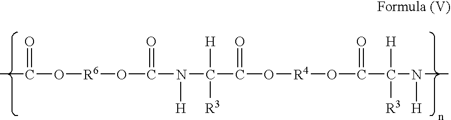
and wherein n ranges from about 5 to about 150; wherein the R³s within an individual n monomer are independently selected from the group consisting of hydrogen, (C₁-C₆) alkyl, (C₂-C₆) alkenyl, (C₆-C₁₀) aryl(C₁-C₆) alkyl and —(CH₂)₂S(CH₃); R⁴ and R⁶ are each selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols, and combinations thereof;

or a PEUR having a chemical structure described by general structural formula (VI), 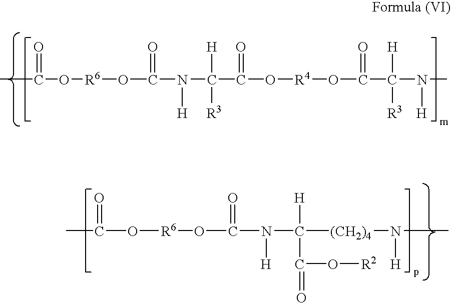
wherein n ranges from about 5 to about 150, m ranges about 0.1 to about 0.9: p ranges from about 0.9 to about 0.1; R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R³s within an individual m monomer are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); R⁴ and R⁶ are each independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols, and combinations thereof;

or a poly(ester urea) (PEU) having a chemical formula described by structural formula (VII), 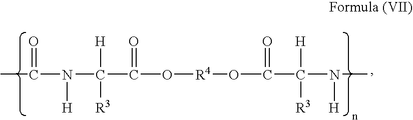
wherein n is about 10 to about 150; the R³s within an individual n monomer are independently selected from the group consisting of hydrogen, (C₁-C₆) alkyl, (C₂-C₆) alkenyl, (C₆-C₁₀) aryl (C₁-C₆)alkyl and —(CH₂)₂S(CH₃); R⁴ is independently selected from the group consisting of (C₂-C₂₀) alkylene, (C₂-C₂₀) alkenylene, (C₂-C₈) alkyloxy (C₂-C₂₀) alkylene, residues of a saturated and unsaturated adhesion preventing diols, bicyclic-fragments of a 1,4:3,6-dianhydrohexitol of structural formula (II) and combinations thereof;

or a PEU having a chemical formula described by structural formula (VIII), 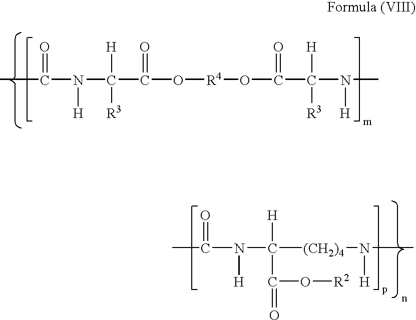
wherein m is about 0.1 to about 1.0; p is about 0.9 to about 0.1; n is about 10 to about 150; each R² is independently selected from the group consisting of hydrogen, (C₁-C₁₂) alkyl, (C₂-C₈) alkyloxy (C₂-C₂₀) alkyl, (C₆-C₁₀) aryl and a protecting group; and the R³s within an individual m monomer are independently selected from the group consisting of hydrogen, (C₁-C₆) alkyl, (C₂-C₆) alkenyl, (C₆-C₁₀) aryl (C₁-C₆)alkyl and —(CH₂)₂S(CH₃); R⁴ is independently selected from the group consisting of (C₂-C₂₀) alkylene, (C₂-C₂₀) alkenylene, (C₂-C₈) alkyloxy (C₂-C₂₀) alkylene, residues of saturated and unsaturated adhesion preventing diols; bicyclic-fragments of a 1,4:3,6-dianhydrohexitol of structural formula (II), and combinations thereof.

In another embodiment, the invention provides methods for applying an adhesion barrier to a tissue surface by applying the invention adhesion barrier composition upon the tissue surface so as to adhere the adhesion barrier to the tissue surface.

In yet another embodiment, the invention provides methods of preventing post-surgical adhesions in a subject undergoing open surgery by applying the invention composition to a tissue surface at a surgical opening so as to form a tissue-adhesive adhesion barrier separating opposing tissue surfaces or tissue-organ surfaces; and closing the surgical opening while maintaining the composition in place for a sufficient time to prevent ingrowths of scar tissue and the formation or reformation of adhesions immediately adjacent to the composition.

### DETAILED DESCRIPTION OF THE INVENTION

The present invention is based on the discovery that thin films of biodegradable polymers and blends thereof that contain amino acids in the polymer chain, such as certain polyester amide (PEA), polyester urethane (PEUR) and polyester urea (PEU) polymers, can be applied to tissue during open surgery to prevent formation of post-surgical adhesions. In certain embodiments, a bioactive agent for adhesion prevention can be dispersed in the polymer of the adhesion barrier for controlled release at the surgical injury site during biodegradation of the adhesion barrier, for example to aid in healing of the wound. The PEA, PEUR and PEU polymers are biodegradable and non-inflammatory and can be used in any combination as a polymer blend in the invention adhesion barrier compositions to achieve desired properties of the polymer.

The invention adhesion barrier composition comprising one or a blend of PEA, PEUR, and PEU polymers can be formulated for two different types of application. In the first embodiment, the adhesion barrier composition is formulated as a solvent-based liquid solution of one or a blend of PEA, PEUR or PEU polymers having a sprayable viscosity. The sprayable liquid composition is applied to the site of a surgical wound as a liquid that forms a tissue-adherent polymer film in situ. The liquid adhesion barrier composition can be applied to tissue by such techniques as spraying, brushing, and the like, to form a film of biodegradable polymer upon the tissue surface to which it is applied. The solvents and solvent mixtures suitable for use in practice of the invention include methanol, ethanol, isopropanol, tetrahydrofuran, methylene chloride, dimethylformamide and dimethyl sulfoxide, and combinations thereof. The preferred solvent and solvent mixtures are those more biocompatible, such as ethanol, isopropanol and dimethyl sulfoxide. The most preferred solvent is ethanol, which is the most biocompatible and volatile solvent.

Both low molecular weight and high molecular weight PEA polymers have been evaluated as ethanol-based formulations for adhesion to tissues of meat and human skin. The solution of low molecular weight PEA in ethanol forms a thin substantially transparent film and adheres strongly to the tissue. “A low weight average molecular weight (M_{w}) polymer” as the term is used herein has M_{w} in the range from about 5,000 Da to about 25,000 Da, for example about 10,000 Da to about 23,000 Da, and defines a polymer that forms a “sticky” film when solvent is evaporated. By contrast, “a high weight average molecular weight (M_{w}) polymer” as the term is used herein has M_{w} in the range from about 85,000 Da to about 300,000 Da, for example about 150,000 Da to about 225,000 Da. A high molecular weight (M_{w}) PEA formulation in ethanol forms a white film on the tissue. Both of these liquid formulations can be effectively used to form a bioabsorbable barrier to formation of post-surgical adhesions.

In another embodiment, the adhesion barrier composition is prepared in the form of a solid polymer film comprising one or a blend of PEA, PEUR, or PEU polymers. Two forms of solid film can be used as a bioabsorbable adhesion barrier. The first form is single layer of thin polymer film, which can be made from a single one or a blend of PEA, PEUR, and PEU polymers. Alternatively, the adhesion barrier can comprise two or more layers of thin films made by solvent casting at least two different PEA, PEUR, or PEU polymers, or blends thereof, that have opposite adhesive properties. For example, a two-layer solid film can be made by solvent casting of two different polymers, or polymer blends, one with high adhesive (e.g., low average molecular weight (M_{w})) and one with low adhesive properties (e.g., low average molecular weight (M_{w})), to form a solid dual-layered composition having, respectively, a sticky side and non-sticky side. In use, the sticky side of the film will adhere to the tissue of a surgical wound and the non-sticky side can be used to prevent adhesion to the adhesion barrier by other tissue. To fabricate a thicker adhesion barrier, multiple layers can be applied.

In one embodiment the invention provides a biodegradable adhesion barrier composition comprising a solution in a biocompatible solvent of a biodegradable polymer, wherein the polymer is selected from at least one of the following: a PEA having a chemical formula described by structural formula (I), 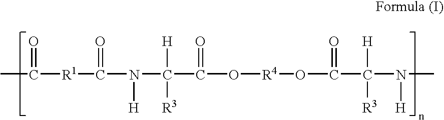
wherein n ranges from about 5 to about 150; R¹ is independently selected from the group consisting of (C₂-C₂₀) alkylene, (C₂-C₂₀) alkenylene, α,ω-bis(4-carboxyphenoxy) (C₁-C₈) alkane, residues of saturated and unsaturated adhesion preventing di-acids, residues of α,ω-alkylene dicarboxylates of formula (III), and combinations thereof; wherein R⁵ and R⁷ in Formula (III) are each independently selected from (C₂-C₁₂) alkylene or (C₂-C₁₂) alkenylene; the R³s in individual n monomers are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing di-acids, and combinations thereof;

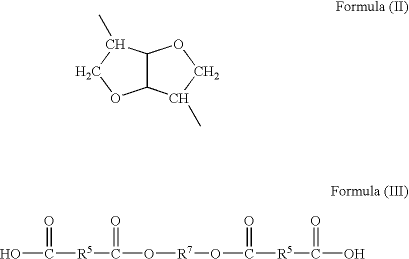

or a PEA having a chemical formula described by structural formula (IV), 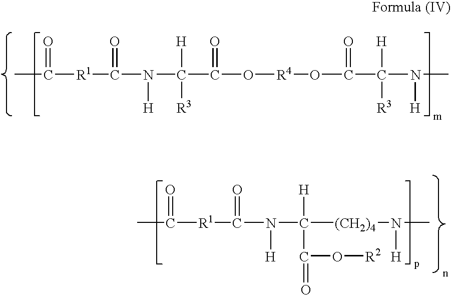
wherein n ranges from about 5 to about 150, m ranges about 0.1 to 0.9: p ranges from about 0.9 to 0.1; wherein R¹ is independently selected from the group consisting of (C₂-C₂₀) alkylene, (C₂-C₂₀) alkenylene, α,ω-bis(4-carboxyphenoxy) (C₁-C₈) alkane, and residues of saturated and unsaturated adhesion preventing di-acids, residues of α,ω-alkylene dicarboxylates of formula (III), and combinations thereof; wherein R⁵ and R⁷ in Formula (III) are each independently selected from (C₂-C₁₂) alkylene or (C₂-C₁₂) alkenylene; each R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R³s in individual m monomers are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols and combinations thereof;

or a poly(ester urethane) (PEUR) having a chemical formula described by structural formula (V), 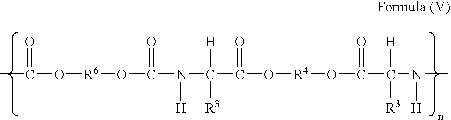
and wherein n ranges from about 5 to about 150; wherein the R³s within an individual n monomer are independently selected from the group consisting of hydrogen, (C₁-C₆) alkyl, (C₂-C₆) alkenyl, (C₆-C₁₀) aryl(C₁-C₆) alkyl and —(CH₂)₂S(CH₃); R⁴ and R⁶ are each selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols, and combinations thereof;

or a PEUR having a chemical structure described by general structural formula (VI), 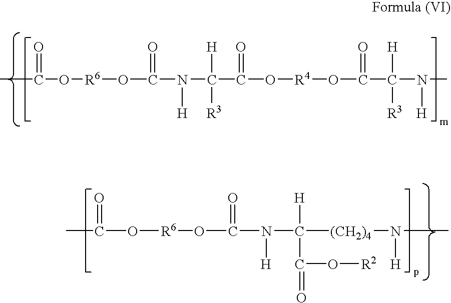
wherein n ranges from about 5 to about 150, m ranges about 0.1 to about 0.9: p ranges from about 0.9 to about 0.1; R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R³s within an individual m monomer are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); R⁴ and R⁶ are each independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols, and combinations thereof.

For example, an effective amount of the residue of at least one adhesion preventing diol, as disclosed herein, can be contained in the polymer backbone. In one alternative in the PEA or PEUR polymer, at least one of R⁴ or R⁶ is a bicyclic fragment of 1,4:3,6-dianhydrohexitol, such as 1,4:3,6-dianhydrosorbitol (DAS).

In still another embodiment the invention adhesion barrier composition can comprise at least one biodegradable PEU polymer having a chemical formula described by structural formula (VII), 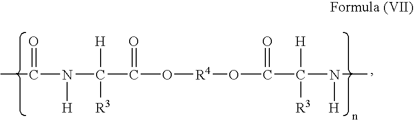
wherein n is about 10 to about 150; the R³s within an individual n monomer are independently selected from the group consisting of hydrogen, (C₁-C₆) alkyl, (C₂-C₆) alkenyl, (C₆-C₁₀) aryl (C₁-C₆)alkyl and —(CH₂)₂S(CH₃); R⁴ is independently selected from the group consisting of (C₂-C₂₀) alkylene, (C₂-C₂₀) alkenylene, (C₂-C₈) alkyloxy (C₂-C₂₀) alkylene, residues of a saturated and unsaturated adhesion preventing diols, bicyclic-fragments of a 1,4:3,6-dianhydrohexitol of structural formula (II) and combinations thereof;

or a PEU having a chemical formula described by structural formula (VIII), 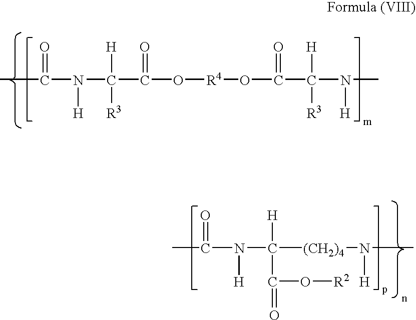
wherein m is about 0.1 to about 1.0; p is about 0.9 to about 0.1; n is about 10 to about 150; each R² is independently selected from the group consisting of hydrogen, (C₁-C₁₂) alkyl, (C₂-C₈) alkyloxy (C₂-C₂₀) alkyl, (C₆-C₁₀) aryl and a protecting group; and the R³s within an individual m monomer are independently selected from the group consisting of hydrogen, (C₁-C₆) alkyl, (C₂-C₆) alkenyl, (C₆-C₁₀) aryl (C₁-C₆)alkyl and —(CH₂)₂S(CH₃); R⁴ is independently selected from the group consisting of (C₂-C₂₀) alkylene, (C₂-C₂₀) alkenylene, (C₂-C₈) alkyloxy (C₂-C₂₀) alkylene, residues of saturated and unsaturated adhesion preventing diols; bicyclic-fragments of a 1,4:3,6-dianhydrohexitol of structural formula (II), and combinations thereof.

For example, an effective amount of the residue of at least one adhesion preventing bioactive agent that is a diol or a diacid, can be contained in the polymer backbone. In one alternative in the PEU polymer, at least one R⁴ is a residue of a saturated or unsaturated adhesion preventing diol, or a bicyclic fragment of a 1,4:3,6-dianhydrohexitol, such as DAS. In yet another alternative in the PEU polymer, at least one R⁴ is a bicyclic fragment of a 1,4:3,6-dianhydrohexitol, such as DAS.

These PEU polymers can be fabricated as high molecular weight polymers useful for making the invention adhesion barrier compositions, and such compositions containing adhesion preventing bioactive agents for delivery to humans and other mammals. PEUs incorporate hydrolytically cleavable ester groups and non-toxic, naturally occurring monomers that contain α-amino acids in the polymer chains. The ultimate biodegradation products of PEUs will be amino acids, diols, and CO₂. In contrast to the PEAs and PEURs, the PEUs are crystalline or semi-crystalline and possess advantageous mechanical, chemical and biodegradation properties that allow formulation of completely synthetic, and hence easy to produce, crystalline and semi-crystalline polymers. For example, the PEU polymers used in the invention adhesion barrier compositions have high mechanical strength, and surface erosion of the PEU polymers can be catalyzed by enzymes present in physiological conditions, such as in the presence of hydrolases.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, or a mixture thereof.

As used herein, an “adhesion preventing diol” means any diol molecule, whether synthetically produced, or naturally occurring (e.g., endogenously) that affects a biological process in a mammalian individual, such as a human, in a therapeutic or palliative manner when administered to the mammal.

As used herein, the term “residue of an adhesion preventing diol” means a portion of an adhesion preventing diol, as described herein, which portion excludes the two hydroxyl groups of the diol. As used herein, the term “residue of an adhesion preventing di-acid” means a portion of an adhesion preventing di-acid, as described herein, which portion excludes the two carboxyl groups of the di-acid. The corresponding adhesion preventing diol or di-acid containing the “residue” thereof is used in synthesis of the polymer compositions. The residue of the adhesion preventing di-acid or diol is reconstituted in vivo (or under similar conditions of pH, aqueous media, and the like) to the corresponding di-acid or diol upon release from the backbone of the polymer by biodegradation in a controlled manner that depends upon the properties of the PEA, PEUR or PEU polymer(s) selected to fabricate the composition, which properties are as known in the art and as described herein.

As used herein the term “adhesion preventing bioactive agent” means a therapeutic or analgesic agent useful in promoting post-operative healing and/or combating formation of adhesions. One or more such adhesion preventing bioactive agents optionally may be dispersed in the invention adhesion barrier compositions. As used herein, the term “dispersed” means that the adhesion preventing bioactive agent is dispersed, mixed, dissolved, homogenized, and/or covalently bound to (“dispersed”) in a polymer, for example attached to a functional group in the biodegradable polymer of the composition. Adhesion preventing bioactive agents, as disclosed herein, that are also adhesion preventing diols or di-acids may optionally be incorporated into the backbone of a PEA, PEUR, or PEU polymer (as a residue thereof). Adhesion preventing bioactive agents may include, without limitation, small molecule drugs, peptides, proteins, DNA, cDNA, RNA, sugars, lipids and whole cells.

The term, “biodegradable” as used herein to describe the PEA, PEUR and PEU polymers, including mixtures and blends thereof, used in fabrication of invention adhesion barrier compositions means the polymer(s) are capable of being broken down in situ into innocuous products in the normal functioning of the body. This is particularly true when the amino acids used in fabrication of the polymers are biological L-α-amino acids. A “biodegradable polymer” as the term is used herein also means the polymer is degraded by water and/or by enzymes found in tissues of mammalian patients, such as humans. The invention adhesion barrier compositions are also suitable for use in veterinary treatment of a variety of mammalian patients, such as pets (for example, cats, dogs, rabbits, ferrets), farm animals (for example, swine, horses, mules, dairy and meat cattle) and race horses when used as described herein.

The term “controlled” as used herein to described the release of adhesion preventing bioactive agent(s) from invention adhesion barrier compositions means the composition biodegrades over a desired period of time, for example from about 3 to about 6 months, for example from about 30 days to about 3 months, depending upon the polymer or polymer mixture used, the thickness of the barrier film or layer used and the structural form of the barrier. In embodiments where the adhesion barrier comprises one or more adhesion preventing bioactive agents, biodegradation of the composition provides a smooth and regular (i.e. “controlled”) time release profile (e.g., avoiding an initial irregular spike in drug release and providing instead a substantially smooth rate of change of release throughout biodegradation of the invention composition).

The polymers in the invention adhesion barrier compositions include hydrolyzable ester and enzymatically cleavable amide linkages that provide biodegradability, and are typically chain terminated, predominantly with amino groups. Optionally, the amino termini of the polymers can be acetylated or otherwise capped by conjugation to any other acid-containing, biocompatible molecule, to include without restriction organic acids, bioinactive biologics, and adhesion preventing bioactive agents as described herein. In one embodiment, the entire polymer composition, and any adhesion barriers made thereof, is substantially biodegradable.

In one alternative, at least one of the α-amino acids used in fabrication of the polymers used in the invention adhesion barrier compositions is a biological α-amino acid. For example, when the R³s are CH₂Ph, the biological O-amino acid used in synthesis is L-phenylalanine. In alternatives wherein the R³s are CH₂—CH(CH₃)₂, the polymer contains the biological α-amino acid, L-leucine. By varying the R³s within monomers as described herein, other biological α-amino acids can also be used, e.g., glycine (when the R³s are H), alanine (when the R³s are CH₃), valine (when the R³s are CH(CH₃)₂), isoleucine (when the R³s are CH(CH₃)—CH₂—CH₃), phenylalanine (when the R³s are CH₂—C₆H₅), or methionine (when the R³s are —(CH₂)₂S(CH₃), and combinations thereof. In yet another alternative embodiment, all of the various α-amino acids contained in the polymers used in making the invention adhesion barrier compositions are biological O-amino acids, as described herein.

The terms, “biodegradable” and “bioabsorbable” as used herein to describe the polymers used in the invention adhesive barrier composition means the polymer is capable of being broken down into innocuous products in the normal functioning of the body. In one embodiment, the entire adhesive barrier is biodegradable. The biodegradable polymers described herein have hydrolyzable ester and enzymatically cleavable amide linkages that provide the biodegradability, and are typically chain terminated, predominantly with amino groups. Optionally, these amino termini can be acetylated or otherwise capped by conjugation to any other acid-containing, biocompatible molecule, to include without restriction organic acids, bioinactive biologics and bioactive compounds

Delivery of Drugs and Biologics

The invention bioabsorbable adhesion barriers compositions can, optionally, comprise one or more adhesion preventing bioactive agent for adhesion prevention, including drugs and biologics, incorporated therein. Such adhesion preventing bioactive agents can be either dissolved or dispersed in the solvent-based polymer formulations. The bioactive agent can also be covalently conjugated to polymers used in the invention compositions. Such adhesion preventing bioactive agents can, optionally, also be incorporated into invention solid layer adhesion barrier compositions, which are made using solvent cast, melt process, or any other appropriate processing method for making solid polymer films or thin layers known in the art.

Adhesion preventing bioactive agents suitable for use in the invention compositions and methods include, without limitation, compounds that have been widely studied for adhesion prevention, such as non-steroidal anti-inflammatory drugs (NSAIDs), corticosteroids, anti-oxidants, anti-neoplastics and transforming growth factor (TGF-beta I). Examples of non-steroidal anti-inflammatory agents include aspirin, diflunisal, acetaminophen, indomethacin, sulindac, and etodolac. Additional suitable non-steroidal anti-inflammatory agents include femanates, such as, mefanamic acid, meclofenamate, flufenamic acid, tolmetin, ketorolac, diclofenac; proprionic acid derivatives, such as, ibuprofen, naproxen, fenoprofen, flurbiprofen, ketoprofen, and oxaprozin; enolic acid derivatives, such as, piroxicam, meloxicam, and nabumetone; and COX-2 selective inhibitors, such as, celecoxib, valdecoxib, parecoxib, etoricoxib, and lumaricoxib.

Examples of steroidal anti-inflammatory agents suitable for use in the invention include dexamethasone, hydrocortisone, prednisolone, cortisone, hydrocortisone, betamethasone, fludrocortisone, prednisone, methylprednisolone, and triamcinolone. Examples of suitable anti-oxidants include methylene blue, superoxide dismutase and other active oxygen inhibitors. Examples of antineoplastics suitable for use in the invention compositions and methods include natural products and derivatives thereof, such as paclitaxel or analogs or derivatives of paclitaxel, vinca alkaloids, estramustine, alkylating agents, such as, mechlorethamine, cyclophosphamide, mephalan, chlorambucil, altretamine, thiotepa, procarbazine, busulfan, carmustine, streptozocin, dacarbazine, temozolamide, cisplatin, carboplatin, oxaliplatin; antimetabolites, such as, pemetrexed, fluorouracil, cytarabine, gemcitabine, mercaptopurine, and pentostatin; hormone antagonists, such as, mitotane, prednisone, diethylstilbestrol, anatozole, tamoxifen, flutamide, leuprolide, testosterone proprionate, hydroxyprogesterone caproate, and miscellaneous agents, such as, hydroxyurea, tretinoin, arsenic trioxide, imatinib, gelfitinib, bortezonib interferon-alfa, and interleukin-2.

Adhesion preventing bioactive agents suitable for inclusion in the invention compositions and methods of use also include, for example, antiproliferants, antifungals, antimicrobials, antiviral agents and opioids.

Suitable examples of antiproliferants include sirolimus, everolimus, mycophenolate mofetil, methotrexate, cyclophosphamide, thalidomide, chlorambucil, and leflunomide. Suitable examples of antifungals include flucytosine, amphoterecin B, fluconazole, itraconazole, voriconazole, butoconazole, clortrimazole, miconazole, nystatin, terconazole, tioconazole, ciclopirox, econazole, ketoconazole, haloprogin, naftifine, oxiconazole, sertaconazole, sulconazole, terbinafine, tolnaftate, undecylenate, griseofulvin, capsofumgin acetate, and benzoic acid and salicylic acid combinations.

Suitable examples of antimicrobials include sulfonamide derivatives, such as, sulfanilamide, sulfamethoxazole, sulfacetamide, sulfadiazine, sulfisoxazole, paraaminobenzoic acid, trimethoprim, quinolone derivatives, such as, nalidixic acid, cinoxacin, norfloxacin, ciprofloxacin, ofloxacin, sparfloxacin, fleroxacin, perfloxacin, levofloxacin, garenoxacin, and gemifloxacin. Additional examples include nitrofurantoin, penicillin derivatives, such as, penicillin G, penicillin V, methicillin, oxacillin, dicloxacillin, nafcillin, ampicillin, amoxicillin, carbenacillin, carbenacillin indanyl, ticarcicllin, mezlocillin, piperacillin, cephalosporin derivatives, such as, cefazolin, cephalexin, cefadroxil, cefaclor, cefprozil, cefuroxime, cefuroxime acetil, loracarbef, cefotetan, ceforanide, cefotaxime, cefpodoxime proxetil, cefibuten, cefnidir, cefditoren pivoxil, ceftizoxime, cefoperazone, ceftazidime, cefepime; carbapenem derivatives, such as, imipenem, meropenem, ertapenem, aztreonam; □-lactamase inhibitors, such as, clavulanic acid, sulbactam, and tazobactam. Aminoglycoside derivatives include; neomycin B, kanamycin A, streptomycin, gentamcin C, tobramycin, netilmicin, amikacin; tetracycline derivatives, such as, tetracycline, chlortetracycline, oxytetracycline, doxycycline, minocycline, methacycline, demeclocycline; and chloramphenicol. Macrolide antimicrobials include: erythromycin, clarithromycin, azithromycin, ketolide derivatives, such as, telithromycin, and amino acid trans-L-4-n-propylhygrinic acid derivatives, such as, clindamycin. Miscellaneous antibacterials are; pristinamycin derivatives, such as, quinupristin and dalforpristin; oxazolidinone derivatives, such as, linezolid. Others include spectinomycin, polymyxin B and colistin, vacomycin, teicoplanin, daptomycin, bacitracin, and mupirocin. Examples of drugs for treating mycobacteriums include dapsone, cycloserine, aminosalicylic acid, ethionamide, linezolid, intereron-C, isoniazid, rifampin, ethambutol, pyrazinamide, capreomycin.clofazimine, and rifabutin. Examples of antiviral agents include acyclovir, cidofovir, famciclovir, foscamet, fomivirsen, ganciclovir, idoxuridine, penciclovir, entecavir, clevudine, emtricitabine, telbivudine, tenofovir, viramidine, resiquimod, maribavir, pleconaril, peramivir, trifluridine; valacyclovir, valganciclovir, amantadine, oseltamivir, rimantadine, zanamiivir, adefovir dipivoxil, interferon-alpha, lamivudine, ribavirin, imiquimod, zidovudine, didanosine, stavudine, zalcitabine, lamivudine, abacavir, tenofovir disoproxil, emtricitabine, nevirapine, efavirenz, delavirdine, saquinavir, indiavir, ritonavir, nelfinavir, amprenavir, lopinavir, atazanavir, fosamprenavir, and enfuvirtide.

Examples of opioid analgesics include morphine, etorphine, codeine, fentanyl, sufentanil, alfentanil, hydromorphone, hydrocodone, levorphanol, meperidine, methadone, oxycodone, oxymorphone, propoxyphene, tramadol, including opioid agonist-antagonist or partial agonist; buprenorphine, butorphanol, nalbuphine, pentazocine, nalorphine, naloxonazine, bremazocine, ethylketocyclazocine, spiradoline, nor-binaltorphimine, naltrindole. Endogenous peptides suitable for use as adhesion preventing bioactive agents in the invention compositions and methods include; met-enkephalin, leu-enkephalin, □-endorphin, Dynorphin A, Dynorphin B, and □-Neoendorphin.

Where the adhesion preventing bioactive agent is a diol or diacid, a residue of such bioactive diol or diacid optionally can be incorporated into the backbone of the polymer for release as a reconstituted adhesion preventing bioactive agent upon biodegradation of the polymer backbone in the invention composition.

The chemical and therapeutic properties of the above described adhesion preventing bioactive agents as inhibitors of post-operative adhesions, antibiotics, and the like, are well known in the art and detailed descriptions thereof can be found, for example, in the 13th Edition of The Merck Index (Whitehouse Station, N.J., USA).

The PEA, PEUR and PEU polymers used in practice of the invention bear functionalities that allow facile covalent attachment to the polymer of an adhesion preventing bioactive agent. For example, a polymer bearing carboxyl groups can readily react with an amino moiety, thereby covalently bonding a peptide to the polymer via the resulting amide group. As will be described herein, the biodegradable polymer and the adhesion preventing bioactive agent may contain numerous complementary functional groups that can be used to covalently attach an adhesion preventing bioactive agent to the biodegradable polymer.

In addition, the polymers disclosed herein (e.g., those having structural formulas (I and IV-VIII), upon enzymatic degradation, provide amino acids while the other breakdown products can be metabolized in the way that fatty acids and sugars are metabolized. Uptake of the polymer with adhesion preventing bioactive agent is safe: studies have shown that the subject can metabolize/clear the polymer degradation products. These polymers and the invention adhesion barrier compositions are, therefore, substantially non-inflammatory to the subject.

The biodegradable PEA, PEUR and PEU polymers useful in forming the invention adhesion barrier compositions may contain multiple different α-amino acids in a single polymer molecule, for example, at least two different amino acids per repeat unit, or a single polymer molecule may contain multiple different α-amino acids in the polymer molecule, depending upon the size of the molecule.

In addition, the polymers used in the invention adhesion barrier compositions display minimal hydrolytic degradation when tested in a saline (PBS) medium, but in an enzymatic solution, such as chymotrypsin or CT, a uniform erosive behavior has been observed.

Suitable protecting groups for use in the PEA, PEUR and PEU polymers include t-butyl or another as is known in the art. Suitable 1,4:3,6-dianhydrohexitols of general formula (II) include those derived from sugar alcohols, such as D-glucitol, D-mannitol, or L-iditol. Dianhydrosorbitol is the presently preferred bicyclic fragment of a 1,4:3,6-dianhydrohexitol for use in the PEA, PEUR and PEU polymers used in fabrication of the invention adhesion barrier compositions.

The term “aryl” is used with reference to structural formulae herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulae herein to mean a divalent branched or unbranched hydrocarbon chain containing at least one unsaturated bond in the main chain or in a side chain.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF), N,N-dimethylformamide (DMF) or N,N-dimethylacetamide (DMAc) is used as the eluent (1.0 mL/min). Polystyrene or poly(methyl methacrylate) standards having narrow molecular weight distribution were used for calibration.

Methods for making polymers of structural formulas containing a α-amino acid in the general formula are well known in the art. For example, for the embodiment of the polymer of structural formula (I) wherein R⁴ is incorporated into an α-amino acid, for polymer synthesis the α-amino acid with pendant R³ can be converted through esterification into a bis-α,ω)-diamine, for example, by condensing the α-amino acid containing pendant R³ with a diol HO—R⁴—OH. As a result, di-ester monomers with reactive α,ω-amino groups are formed. Then, the bis-α,ω-diamine is entered into a polycondensation reaction with a di-acid such as sebacic acid, or bis-activated esters, or bis-acyl chlorides, to obtain the final polymer having both ester and amide bonds (PEA). Alternatively, for example, for polymers of structure (I), instead of the di-acid, an activated di-acid derivative, e.g., bis-para-nitrophenyl diester, can be used as an activated di-acid. Additionally, a bis-di-carbonate, such as bis(p-nitrophenyl) dicarbonate, can be used as the activated species to obtain polymers containing a residue of a di-acid. In the case, of PEUR polymers, a final polymer is obtained having both ester and urethane bonds.

More particularly, synthesis of the unsaturated poly(ester-amide)s (UPEAs) useful as biodegradable polymers of the structural formula (I) as disclosed above will be described, wherein 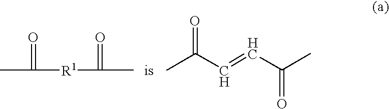
and/or (b) R⁴ is —CH₂—CH═CH—CH₂—. In cases where (a) is present and (b) is not present, R⁴ in (I) is —C₄H₈— or —C₆H₁₂—. In cases where (a) is not present and (b) is present, R¹ in (I) is —C₄H₈— or —C₈H₁₆—.

The UPEAs can be prepared by solution polycondensation of either (1) di-p-toluene sulfonic acid salt of bis(α-amino acid) di-ester of unsaturated diol and di-p-nitrophenyl ester of saturated dicarboxylic acid or (2) di-p-toluene sulfonic acid salt of bis(α-amino acid) diester of saturated diol and di-nitrophenyl ester of unsaturated dicarboxylic acid or (3) di-p-toluene sulfonic acid salt of bis(α-amino acid) diester of unsaturated diol and di-nitrophenyl ester of unsaturated dicarboxylic acid.

The aryl sulfonic acid salts of diamines are known for use in synthesizing polymers containing amino acid residues. The p-toluene sulfonic acid salts are used instead of the free diamines because the aryl sulfonic salts of bis(α-amino acid) diesters are easily purified through recrystallization and render the amino groups as less reactive ammonium tosylates throughout workup. In the polycondensation reaction, the nucleophilic amino group is readily revealed through the addition of an organic base, such as triethylamine, reacts with bis-electrophilic monomer, so the polymer product is obtained in high yield.

Bis-electrophilic monomer, for example, the di-p-nitrophenyl esters of unsaturated dicarboxylic acid can be synthesized from p-nitrophenyl and unsaturated dicarboxylic acid chloride, e.g., by dissolving triethylamine and p-nitrophenol in acetone and adding unsaturated dicarboxylic acid chloride dropwise with stirring at −78° C. and pouring into water to precipitate product. Suitable acid chlorides included fumaric, maleic, mesaconic, citraconic, glutaconic, itaconic, ethenyl-butane dioic and 2-propenyl-butanedioic acid chlorides. For polymers of structure (V) and (VI), bis-p-nitrophenyl dicarbonates of saturated or unsaturated diols are used as the activated monomer. Dicarbonate monomers of general structure (IX) are employed for polymers of structural formula (V) and (VI), 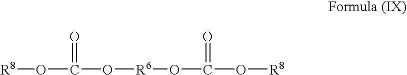
wherein each R⁵ is independently (C₆-C₁₀) aryl optionally substituted with one or more nitro, cyano, halo, trifluoromethyl, or trifluoromethoxy; and R⁸ is independently (C₂-C₂₀) alkylene, (C₂-C₂₀) alkyloxy, or (C₂-C₂₀) alkenylene.

Suitable adhesion preventing diol compounds that can be used to prepare bis(α-amino acid) diesters of adhesion preventing diol monomers, or bis(carbonate) of adhesion preventing di-acid monomers, for introduction into the invention adhesion barrier compositions include naturally occurring adhesion preventing diols, such as 17-β-estradiol, a natural and endogenous hormone. The procedure for incorporation of an adhesion preventing diol, as disclosed herein, into the backbone of a PEA, PEUR or PEU polymer is illustrated in this application by Example 8, in which active steroid hormone 17-β-estradiol containing mixed hydroxyls—secondary and phenolic—is introduced into the backbone of a PEA polymer. When the PEA polymer is used to fabricate adhesion barrier compositions and the adhesion barrier compositions are implanted in vivo, e.g., during open surgery, the adhesion preventing diol is released from the implanted adhesion barrier at a controlled rate.

Due to the versatility of the PEA, PEUR and PEU polymers used in the invention compositions, the amount of the adhesion preventing diol or di-acid incorporated in a polymer backbone can be controlled by varying the proportions of the building blocks of the polymer. For example, depending on the composition of the PEA, loading of up to 40% w/w of 17β-estradiol can be achieved. Two different regular, linear

PEAs with various loading ratios of 17β-estradiol illustrate this concept in Scheme 1 below:

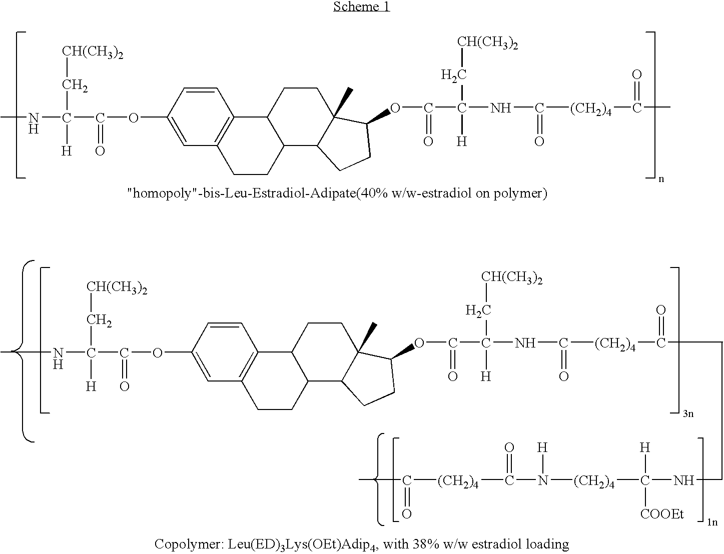

The di-aryl sulfonic acid salts of diesters of α-amino acid and unsaturated diol can be prepared by admixing α-amino acid, e.g., p-aryl sulfonic acid monohydrate and saturated or unsaturated diol in toluene, heating to reflux temperature, until water evolution is minimal, then cooling. The unsaturated diols include, for example, 2-butene-1,3-diol and 1,18-octadec-9-en-diol.

Saturated di-p-nitrophenyl esters of dicarboxylic acid and saturated di-p-toluene sulfonic acid salts of bis-α-amino acid esters can be prepared as described in U.S. Pat. No. 6,503,538 B1.

Synthesis of the unsaturated poly(ester-amide)s (UPEAs) useful as biodegradable polymers of the structural formula (I) as disclosed above will now be described. UPEAs having the structural formula (I) can be made in similar fashion to the compound (VII) of U.S. Pat. No. 6,503,538 B1, except that R⁴ of (III) of U.S. Pat. No. 6,503,538 and/or R¹ of (V) of U.S. Pat. No. 6,503,538 is (C₂-C₂₀) alkenylene as described above. The reaction is carried out, for example, by adding dry triethylamine to a mixture of said (III) and (IV) of U.S. Pat. No. 6,503,538 and said (V) of U.S. Pat. No. 6,503,538 in dry N,N-dimethylacetamide, at room temperature, then increasing the temperature to 80° C. and stirring for 16 hours, then cooling the reaction solution to room temperature, diluting with ethanol, pouring into water, separating polymer, washing separated polymer with water, drying to about 30° C. under reduced pressure and then purifying up to negative test on p-nitrophenol and p-toluene sulfonate. A preferred reactant (IV) of U.S. Pat. No. 6,503,538 is p-toluene sulfonic acid salt of Lysine benzyl ester, the benzyl ester protecting group is preferably removed from (II) to confer biodegradability, but it should not be removed by hydrogenolysis as in Example 22 of U.S. Pat. No. 6,503,538 because hydrogenolysis would saturate the desired double bonds; rather the benzyl ester group should be converted to an acid group by a method that would preserve unsaturation. Alternatively, the lysine reactant (IV) of U.S. Pat. No. 6,503,538 can be protected by a protecting group different from benzyl that can be readily removed in the finished product while preserving unsaturation, e.g., the lysine reactant can be protected with t-butyl (i.e., the reactant can be t-butyl ester of lysine) and the t-butyl can be converted to H while preserving unsaturation by treatment of the product (II) with acid.

In unsaturated compounds having either structural formula (I) or (V), the following hold. An amino substituted aminoxyl (N-oxide) radical bearing group, e.g., 4-amino TEMPO, can be attached using carbonyldiimidazol, or suitable carbodiimide, as a condensing agent. Adhesion preventing bioactive agents, as described herein, can be attached via the double bond functionality. Hydrophilicity can be imparted by bonding to poly(ethylene glycol) diacrylate.

The biodegradable PEA, PEUR and PEU polymers can contain from one to multiple different α-amino acids per polymer molecule and preferably have weight average molecular weights ranging from 5,000 to 300,000. These polymers and copolymers typically have intrinsic viscosities at 25° C., as determined by standard viscosimetric methods, ranging from 0.1 to 4.0, for example, ranging from 0.3 to 3.5.

PEA and PEUR polymers contemplated for use in the practice of the invention can be synthesized by a variety of methods well known in the art. For example, tributyltin (IV) catalysts are commonly used to form polyesters such as poly(ε-caprolactone), poly(glycolide), poly(lactide), and the like. However, it is understood that a wide variety of catalysts can be used to form polymers suitable for use in the practice of the invention.

Such poly(caprolactones) contemplated for use have an exemplary structural formula (X) as follows:

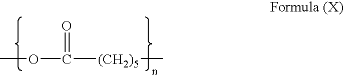

Poly(glycolides) contemplated for use have an exemplary structural formula (XI) as follows:

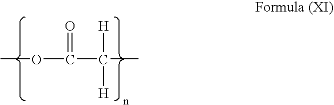

Poly(lactides) contemplated for use have an exemplary structural formula (XII) as follows:

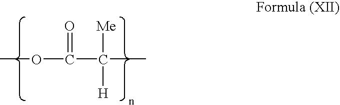

An exemplary synthesis of a suitable poly(lactide-co-ε-caprolactone) including an aminoxyl moiety is set forth as follows. The first step involves the copolymerization of lactide and ε-caprolactone in the presence of benzyl alcohol using stannous octoate as the catalyst to form a polymer of structural formula (XIII).

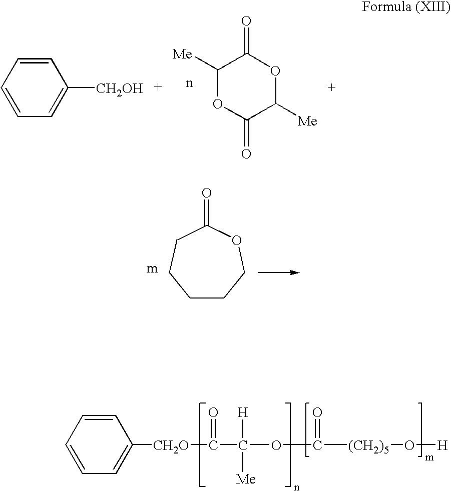

The hydroxy terminated polymer chains can then be capped with maleic anhydride to form polymer chains having structural formula (XIV):

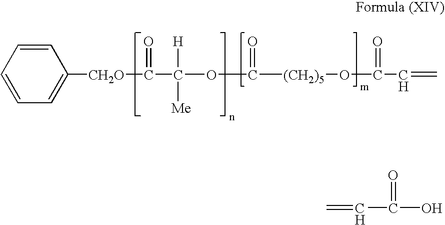

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

In unsaturated compounds having structural formula (VII) for PEU, the following hold: An amino substituted aminoxyl (N-oxide) radical bearing group e.g., 4-amino TEMPO, can be attached using carbonyldiimidazole, or suitable carbodiimide, as a condensing agent. Additional adhesion preventing bioactive agents, and the like, as described herein, optionally can be attached via the double bond functionality provided that the adhesion preventing diol residue in the polymer composition does not contain a double or triple bond.

For example, the invention high molecular weight semi-crystalline PEUs having structural formula (VII) can be prepared inter-facially by using phosgene as a bis-electrophilic monomer in a chloroform/water system, as shown in the reaction scheme (2) below:

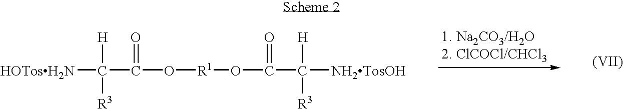

Synthesis of copoly(ester ureas) (PEUs) containing L-Lysine esters and having structural formula (VIII) can be carried out by a similar scheme (3):

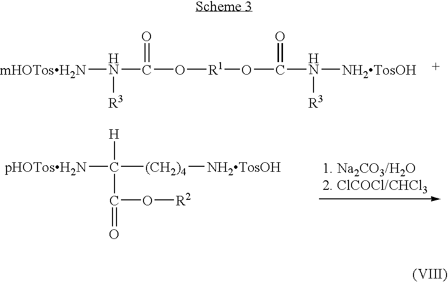

A 20% solution of phosgene (ClCOCl) (highly toxic) in toluene, for example (commercially available (Fluka Chemie, GMBH, Buchs, Switzerland), can be substituted either by diphosgene (trichloromethylchloroformate) or triphosgene (bis(trichloromethyl)carbonate). Less toxic carbonyldiimidazole can be also used as a bis-electrophilic monomer instead of phosgene, di-phosgene, or tri-phosgene.

General Procedure for Synthesis of PEUs It is necessary to use cooled solutions of monomers to obtain PEUs of high molecular weight. For example, to a suspension of di-p-toluenesulfonic acid salt of bis(α-amino acid)-α,ω-alkylene diester in 150 mL of water, anhydrous sodium carbonate is added, stirred at room temperature for about 30 minutes and cooled to about 2-0° C., forming a first solution. In parallel, a second solution of phosgene in chloroform is cooled to about 15-10° C. The first solution is placed into a reactor for interfacial polycondensation and the second solution is quickly added at once and stirred briskly for about 15 min. Then chloroform layer can be separated, dried over anhydrous Na₂SO₄, and filtered. The obtained solution can be stored for further use.

All the exemplary PEU polymers fabricated were obtained as solutions in chloroform and these solutions are stable during storage. However, some polymers, for example, 1-Phe-4, become insoluble in chloroform after separation. To overcome this problem, polymers can be separated from chloroform solution by casting onto a smooth hydrophobic surface and allowing chloroform to evaporate to dryness. No further purification of obtained PEUs is needed. The yield and characteristics of exemplary PEUs obtained by this procedure are summarized in Table 1 herein.

General Procedure for Preparation of porous PEUs. Methods for making the PEU polymers containing α-amino acids in the general formula will now be described. For example, for the embodiment of the polymer of formula (I) or (III), the α-amino acid can be converted into a bis(α-amino acid)-α,ω-diol-diester monomer, for example, by condensing the α-amino acid with a diol HO—R¹—OH. As a result, ester bonds are formed. Then, acid chloride of carbonic acid (phosgene, diphosgene, triphosgene) is entered into a polycondensation reaction with a di-p-toluenesulfonic acid salt of a bis(α-amino acid)-alkylene diester to obtain the final polymer having both ester and urea bonds. In the present invention, at least one adhesion preventing diol can be used in the polycondensation protocol.

The unsaturated PEUs can be prepared by interfacial solution condensation of di-p-toluenesulfonate salts of bis(α-amino acid)-alkylene diesters, comprising at least one double bond in R¹. Unsaturated diols useful for this purpose include, for example, 2-butene-1,4-diol and 1,18-octadec-9-en-diol. Unsaturated monomer can be dissolved prior to the reaction in alkaline water solution, e.g. sodium hydroxide solution. The water solution can then be agitated intensely, under external cooling, with an organic solvent layer, for example chloroform, which contains an equimolar amount of monomeric, dimeric or trimeric phosgene. An exothermic reaction proceeds rapidly, and yields a polymer that (in most cases) remains dissolved in the organic solvent. The organic layer can be washed several times with water, dried with anhydrous sodium sulfate, filtered, and evaporated. Unsaturated PEUs with a yield of about 75%-85% can be dried in vacuum, for example at about 45° C.

To obtain a porous, strong PEU material, L-Leu based PEUs, such as 1-L-Leu-4 and 1-L-Leu-6, can be fabricated using the general procedure described below. Such procedure is less successful in formation of a porous, strong material when applied to L-Phe based PEUs.

The reaction solution or emulsion (about 100 mL) of PEU in chloroform, as obtained just after interfacial polycondensation, is added dropwise with stirring to 1,000 mL of about 80° C.-85° C. water in a glass beaker, preferably a beaker made hydrophobic with dimethyldichlorsilane to reduce the adhesion of PEU to the beaker's walls. The polymer solution is broken in water into small drops and chloroform evaporates rather vigorously. Gradually, as chloroform is evaporated, small drops combine into a compact tar-like mass that is transformed into a sticky rubbery product. This rubbery product is removed from the beaker and put into hydrophobized cylindrical glass-test-tube, which is thermostatically controlled at about 80° C. for about 24 hours. Then the test-tube is removed from the thermostat, cooled to room temperature, and broken to obtain the polymer. The obtained porous bar is placed into a vacuum drier and dried under reduced pressure at about 80° C. for about 24 hours. In addition, any procedure known in the art for obtaining porous polymeric materials can also be used.

Properties of high-molecular-weight porous PEUs made by the above procedure yielded results as summarized in Table 2.

TABLE 1
Properties of PEU Polymers of Formula (VII) and (VIII)

| | | η_{red}^{a)} | | | | Tg^{c)} | Tₘ^{c)} |
|---|---|---|---|---|---|---|---|
| PEU* | Yield [%] | [dL/g] | M_{w}^{b)} | Mₙ^{b)} | M_{w}/Mₙ^{b)} | [° C.] | [° C.] |
| 1-L-Leu-4 | 80 | 0.49 | 84000 | 45000 | 1.90 | 67 | 103 |
| 1-L-Leu-6 | 82 | 0.59 | 96700 | 50000 | 1.90 | 64 | 126 |
| 1-L-Phe-6 | 77 | 0.43 | 60400 | 34500 | 1.75 | — | 167 |
| [1-L-Leu-6]_{0.75}-[1-L- | 84 | 0.31 | 64400 | 43000 | 1.47 | 34 | 114 |
| Lys(OBn)]_{0.25} | | | | | | | |
| 1-L-Leu-DAS | 57 | 0.28 | 55700^{d)} | 27700^{d)} | 2.1^{d)} | 56 | 165 |

*PEUs of general formula (VIII), where, 1-L-Leu-4: R⁴ = (CH₂)₄, R³ = i-C₄H₉1-L-Leu-6: R⁴ = (CH₂)₆, R³ = i-C₄H₉1-L-Leu-6: .R⁴ = (CH₂)₆, R³ = —CH₂—C₆H₅.
1-L-Leu-DAS: R⁴ = 1,4:3,6-dianhydrosorbitol, R³ = i-C₄H
^{a)}Reduced viscosities were measured in DMF at 25° C. and a concentration 0.5 g/dL
^{b)}GPC Measurements were carried out in DMF, (PMMA)
^{c)}Tg taken from second heating curve from DSC Measurements (heating rate10° C./min).
^{d)}GPC Measurements were carried out in DMAc, (PS)

Tensile strength of illustrative synthesized PEUs was measured and results are summarized in Table 2. Tensile strength measurement was obtained using dumbbell-shaped PEU films (4×1.6 cm), which were cast from chloroform solution with average thickness of 0.125 mm and subjected to tensile testing on tensile strength machine (Chatillon TDC200) integrated with a PC using Nexygen FM software (Amtek, Largo, Fla.) at a crosshead speed of 60 mm/min. Examples illustrated herein can be expected to have the following mechanical properties:

1. A glass transition temperature in the range from about 30 C.° to about 90 C.°, for example, in the range from about 35 C.° to about 7 C.°;

2. A film of the polymer with average thickness of about 1.6 cm will have tensile stress at yield of about 20 Mpa to about 150 Mpa, for example, about 25 Mpa to about 60 Mpa;

3. A film of the polymer with average thickness of about 1.6 cm will have a percent elongation of about 10% to about 200%, for example about 50% to about 150%; and

4. A film of the polymer with average thickness of about 1.6 cm will have a Young's modulus in the range from about 500 MPa to about 2000 MPa. Table 2 below summarizes the properties of exemplary PEUs of this type.

TABLE 2
Mechanical Properties of PEUs

| | | Tensile Stress | Percent | Young's |
|---|---|---|---|---|
| | Tg^{a)} | at Yield | Elongation | Modulus |
| Polymer designation | (° C.) | (MPa) | (%) | (MPa) |
| 1-L-Leu-6 | 64 | 21 | 114 | 622 |
| [1-L-Leu-6]_{0.75}-[1-L- | 34 | 25 | 159 | 915 |
| Lys(OBn)]_{0.25} | | | | |

Formation of the Adhesion Barrier In Situ

In one embodiment, the invention adhesion barrier composition is prepared as a sprayable solution in a biocompatible solvent of the polymer, optionally containing one or more adhesion preventing bioactive agents dispersed therein. The composition is applied to the target area as a liquid or viscous solution and the adhesion barrier is formed in situ. For example, the composition can be sprayed on the target area. Sprayability largely depends on the viscosity of the solution, which depends on such factors as the characteristics of the polymer, the polymer concentration in the solution, and the average molecular weight (M_{w}) of the polymer, three factors that can be controlled when any of the PEA, PEUR and PEU polymers of Formulas (I and IV-VIII) are used in the formulation. For example, by varying the structure of the polymers within the parameters described in Formulas (I and IV-VIII), a wide variety of polymer characteristics are achievable, including crosslinking, greater or lesser elasticity, greater or lesser adhesion, and the like. Solution viscosities as high as about 100 CP and higher have been successfully sprayed and those of skill in the art will understand that by judicious combination of the above factors the viscosity of the polymer solution can be readily controlled and optimized. In addition, the sprayability of any particular formulation heavily depends on the spray system used, for example, whether a hand pump, such as the Pfeiffer cartridge pump system or an airbrush spray system is used. Therefore, a wide range of polymer solution viscosities can be sprayed, depending on the spraying system used. The sprayability of PEA polymer-ethanol formulations was evaluated by two spray techniques in Example 5 herein. In yet other embodiments, the invention adhesion barrier is formed in situ by applying the polymer composition to the target area by painting on the solution with a brush or other applicator.

Preformed Solid Polymer Films

In other embodiments, the composition is formed into a solid or porous film or film, which is applied to the target area by laying the polymer film or film upon the surgically exposed target area. For example, if a film is used, the film may have any size suitable for application to the target surface, for example, from about 5 mm by 5 mm to about 200 mm to 200 mm with the thickness in the range of 0.01 mm to 0.5 mm.

In yet further embodiments, the invention preformed solid adhesion barrier compositions are preformed as porous solids. A “porous solid” fabrication of the invention polymer compositions, as the term is used herein, means compositions that have a ratio of surface area to volume greater than 1:1. The maximum porosity of an invention solid adhesion barrier composition will depend upon its shape and method of fabrication. Any of the various methods for creating pores in polymers may be used in connection with the present invention. The following examples of methods for fabricating the invention compositions as preformed porous solid films or layers are illustrative and not intended to be limiting.

In the first example, porosity of the composition is achieved after the solid adhesion barrier composition is formed by cutting pores through the solid composition, for example by laser cutting or etching, such as reactive ion etching. For example, short-wavelength UV laser energy is superior to etching for clean-cutting, drilling, and shaping the invention adhesion barrier composition. UV laser technology first developed by Massachusetts Institute of Technology (MIT) allows for removal of very fine and measured amounts of material as a plasma plume by “photo-ablation” with each laser pulse leaving a cleanly-sculpted pore, or channel. The large size characteristic of the UV excimer laser beam allows it to be separated into multiple beamlets through near-field imaging techniques, so that multiple pores, for example, can be simultaneously bored with each laser pulse. Imaging techniques also allow sub-micron resolution so that nano features can be effectively controlled and shaped. For example, micro-machining of thickness of 250 microns and channel depth of 200 microns, with pore depth of 50 microns has been achieved using this technique on Polycarbonate, Polyethylene Terephthalate, and Polyimide. The technique is equally applicable to films of the invention preformed solid adhesion barrier compositions.

In another example, porosity of the invention solid adhesion barrier compositions is achieved by adding a pore-forming substance, such as a gas, or a pore-forming substance (i.e., a porogen) that releases a gas when exposed to heat or moisture, to the polymer dispersions and solutions used in casting or spraying the layers of the invention adhesion barrier composition. Such pore-forming substances are well known in the art. For example, ammonium bicarbonate salt particles evolve ammonia and carbon dioxide within the solidifying polymer matrix upon solvent evaporation. This method results in a product adhesion barrier composition of one or more layers having vacuoles formed therein by gas bubbles. The expansion of pores within the polymer matrix, leads to well interconnected macro-porous pores, for example, having mean pore diameters of around 300-400 μm (Y. S. Nam et al., Journal of Biomedical Materials Research Part B: Applied Biomaterials (2000) 53(1):1-7). Additional techniques known in the art for creating pores in polymers are the combination of solvent-casting with particulate-leaching, and temperature-induced-phase-separation combined with freeze-drying.

In yet another embodiment, a layer of the solid adhesion barrier composition is cast (e.g., spun by electrospinning) as an entanglement of fine polymer fibers onto a substrate or a preceding layer of the composition, such that a polymer mat or pad is formed upon drying of the layer. Electrospinning produces polymer fibers with diameter in the range of 100 nm and even less, from polymer solutions, suspensions of solid particles and emulsions by spinning a droplet in a field of about 1 kV/cm. The electric force results in an electrically charged jet of polymer solution out-flowing from a droplet tip. After the jet flows away from the droplet in a nearly straight line, the droplet bends into a complex path and other changes in shape occur, during which electrical forces stretch and thin the droplet by very large ratios. After the solvent evaporates, solidified macro to nanofibers remain (D. H. Reneker et al. Nanotechnology (1996) 7:216-223).

Those of skill in the art would understand that, in practice, the porosity of the invention solid adhesion barrier composition should be considered in light of the requirement of the composition to serve as an adhesion barrier.

The invention adhesion barriers can be implanted during open surgery to accomplish a variety of goals. For example, invention adhesion barrier compositions the following exemplary purposes:

1. to separate opposing tissues and prevent ingrowth of scar tissues or to prevent formation or reformation of adhesions immediately adjacent to the adhesion barrier.

2. to aid in a re-operation procedure by promoting formation of a surgical dissection plane immediately adjacent to the adhesion barrier.

3. to promote the formation of a surgical dissection plain in the pericardium, epicardium, retrosternal area, peritoneum, peritoneal cavity, bowels, cecum, organs, or in the female pelvic area, reproductive organs, ovaries, uterus, or uterine tube.

4. to reinforce soft tissues where weakness exists, or for the repair of hernia or other fascial defects that require the addition of a reinforcing or bridging material to obtain the desired surgical result.

5. to provide temporary wound support in such procedures as vaginal prolapse repair, colon or rectal prolapse repair, reconstruction of the pelvic floor and colposuspension. The invention also utilizes biodegradable polymer spray- or solid film-mediated delivery techniques to deliver adhesion preventing bioactive agents into a site of tissue injury caused during surgery to any of the above interior body sites.

The following Examples are meant to illustrate and not to limit the invention.

### EXAMPLE 1

This example illustrates preparation of a low molecular weight PEUR of co-poly-8-[Leu(6)_{0.75}][Lys(Bz) 0.25], which is described by structural formula (IV), wherein m=0.75, p=0.25, R¹=(CH₂)₈, R²═CH₂Ph, R³═CH₂CH(CH₃)₂, and R⁴═(CH₂)₆.

For synthesis of the PEUR, triethylamine (NEt₃) (9.51 mL, 0.07 mole) was added to a mixture of di-p-toluenesulfonic acid salt of bis-(L-leucine)-1,6-hexylene diester (16.0237 g, 0.02 mole); di-p-toluenesulfonic acid salt of bis-(L-lysine(Bz)) (4.5025 g, 0.00775 mole) and di-p-nitrophenyl sebacinate (12.4051 g, 0.03 mole) in dimethylformamide (DMF) (13.75 mL) at room temperature. Afterwards, the temperature of the mixture was increased to about 60° C. and stirring continued for about 24 hours. The reaction solution was cooled to room temperature, diluted with DMF (123.72 mL) (total volume of DMF and NEt₃ is 150 mL, concentration of 10% (w/v)). The reaction solution was thoroughly washed with water and sodium bicarbonate (1% w/v). For final purification, the polymer obtained was dissolved in ethanol (150 mL, 10% w/v). The solution was precipitated in ethyl acetate (1.5 L). Precipitation in the ethyl acetate was repeated until a negative test on p-nitrophenol (a by-product of the polycondensation) was obtained, normally 1-2 times.

The obtained polymer was dissolved in ethanol, filtered and dried at about 65° C. under reduced pressure until dry. Yield was about 50%, weight average molecular weight (M_{w})=22,500 (Gel Permeation Chromatography (GPC, PS) in N,N-dimethylacetamide (DMAc)).

### EXAMPLE 2

This example illustrates preparation of a high molecular weight PEUR of co-poly-8-[Leu(6)_{0.75}][Lys(Bz)_{0.25}], which is described by structural formula (IV), wherein m=0.75, p=0.25, R¹=(CH₂)₈, R²═CH₂Ph, R³═CH₂CH(CH₃)₂, and R⁴═(CH₂)₆ For synthesis, triethylamine (NEt₃) (9.51 mL, 0.07 mole) was added to a mixture of di-p-toluenesulfonic acid salt of bis-(L-leucine)-1,6-hexylene diester (16.0237 g, 0.02 mole); di-p-toluenesulfonic acid salt of bis-(L-lysine(Bz)) (4.5025 g, 0.00775 mole) and di-p-nitrophenyl sebacinate (13.7834 g, 0.033 mole) in dimethylformamide (DMF) (16.33 mL) at room temperature. Afterwards, the temperature of the mixture was increased to about 60° C. and stirring continued for about 24 hours. The viscous reaction solution was cooled to room temperature, diluted with DMF (123.72 mL) (total volume of DMF and NEt₃ is 150 mL, concentration of 10% (w/v)). Acetic anhydride (0.567 mL, 0.006 mole) was added and the reaction solution was stirred for about 16 hours. The reaction solution was thoroughly washed with water and sodium bicarbonate (1% w/v). For final purification, the polymer obtained was dissolved in acetone (150 mL, 10% w/v). The solution was precipitated in ether (1.5 L). Precipitation in the ether was repeated until a negative test on p-nitrophenol (a by-product of the polycondensation) was obtained, normally 1-2 times.

The obtained polymer was dissolved in ethanol, filtered and dried at about 65° C. under reduced pressure until dry. Yield was about 80-90%, M_{w}=168,000 (GPC in N,N-dimethylacetamide (DMAc).

### EXAMPLE 3

This example illustrates two methods for applying the invention solvent-based PEA adhesion barrier composition to a surface of meat (fresh cut beef steak from the supermarket).

Method One: 6 g of a low molecular weight PEA 8-Leu(6 (GPC Mw 23,000 Da) was dissolved in 40 mL of reagent grade ethanol (15% wt/v). The resulting polymer-ethanol solution was loaded into a 50 mL Pfeiffer cartridge pump system. Then, the polymer-ethanol solution was sprayed onto the surface of fresh cut meat using the hand pump system. The ethanol solvent was either evaporated or absorbed by the tissue in one or two minutes, and a thin polymer film formed on the meat surface.

Method Two: 7 g of high molecular weight PEA 8-Leu(6) (GPC Mw 168 kDa) was dissolved in 35 mL Reagent grade ethanol (20% wt/v). A sufficient amount of the polymer-ethanol solution was painted onto the surface of the meat using cotton swabs to form a coating. A white polymer film formed in two to three minutes on the surface of the meat. Such a white polymer film can also immediately be formed on the surface of the meat by rinsing the coating with water.

### EXAMPLE 4

This example illustrates the two methods for applying solvent based PEAn adhesion barrier to the skin of a human hand.

Method One: 6 g of a low molecular weight PEA 8-Leu(6) (GPC Mw 23 kDa) was dissolved in 40 ml reagent grade ethanol (15% wt/v). The polymer ethanol solution was sprayed onto the skin of a human hand using a 50 mL Pfeiffer cartridge pump system. A thin physical polymer barrier was formed on the skin after evaporation of the ethanol solvent. Adhesion of the thin polymer film to the skin was so strong that the thin polymer film could not be rubbed off the skin.

Method Two: 7 g of high molecular weight PEA-Leu(6) polymer (GPC Mw 168K) was dissolved in 35 ml of reagent grade ethanol (20% wt/v). A sufficient amount of the polymer-ethanol solution was applied to the skin surface of a human hand using a cotton swab to form a coating. A polymer barrier layer was formed on the skin after evaporation of the ethanol solvent. This polymer barrier could be rubbed or peeled off as a white film after applying a substantial amount of force.

### EXAMPLE 5

This example illustrates the sprayability of PEA polymer solutions. The sprayability of PEA polymer-ethanol formulations was evaluated by two spray techniques. A 50 ml hand pump (cartridge pump system, Pfeiffer Vacuum, Milpitis, Calif.) was the first spray technique evaluated. A low molecular weight polymer (Mw 23 kDa) solution was uniformly sprayed at a PEA polymer concentration up to 20% (wt/v). A high molecular weight PEA polymer (Mw 168 kDa) solution was uniformly sprayed at a polymer concentration of up to 5% (wt/v). Addition of isopropanol (up to 2:1 isopropanol/ethanol ratio) improved the sprayability of the high molecular weight polymer solution.

The sprayability of PEA polymer-ethanol formulations was also evaluated using an airbrush set (Passche Airbrush, Chicago, Ill.). Improved uniformity of spray was achieved with the airbrush equipment.

### EXAMPLE 6

This example illustrates the macrophage mediated degradation of the PEA polymers. The ability of macrophages to degrade PEA was assessed by in vitro culture. For this experiments PEAs of formula (IV) with acetylated end group (AC) and various R² substituents were selected: Bz (benzyl), TEMPO (4-amino-2,2,6,6 tetramethylpiperidine-1-oxyl), dansyl (didansyl-L-lysine). PEA.Ac.Bz, PEA.Ac.TEMPO, and a dansylated-PEA were dissolved in ethanol (10% w/v) and filtered through a 0.45 μm filter. The dansylated polymer is fluorescent and provided a means to visually examine polymer uptake into cells. PEA.Ac.Bz:dansylated-PEA and PEA.Ac.TEMPO:dansylated-PEA blends of 95%:5% (w/w) were mixed and cast into tissue culture polystyrene plates at a concentration of 15 mg/well. The plates were air dried and sterilized by gamma irradiation.

Human monocytes were isolated from healthy donors using density centrifugation and negative magnetic bead selection (Miltenyi Biotec, Auburn, Calif.) and were seeded onto the polymers at a density of 750,000 cells/well. Culture media containing 10% (v/v) autologous serum was added as a negative control. Approximately 1.2-1.5 mL of supernatant, media, or chymotrypsin was collected at days 3, 7, 10, and 14. The samples were frozen at −20° C. until processing.

The samples were concentrated by Speed Vac, and 1 mL of THF was added to precipitate the proteins. The samples were then centrifuged for 5 minutes at 13,000 RPM in a microcentrifuge and the supernatants collected. 600 μl of methanol was added to further precipitate protein, and the samples were again centrifuged. The supernatant was collected and added to the THF supernatant. The supernatants were concentrated by Speed Vac and dried under argon. The samples were then reconstituted with 600 μl of THF and centrifuged for 3-5 minutes at 4,000 RPM. Collected samples (100 μl of the supernatants) were evaluated by gas phase chromatography (GPC).

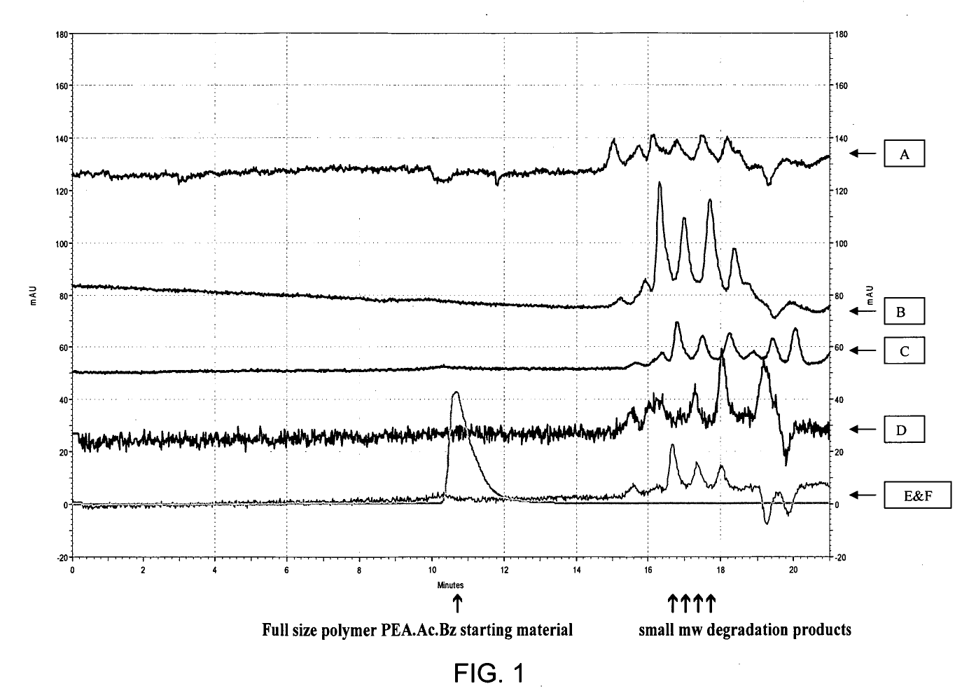

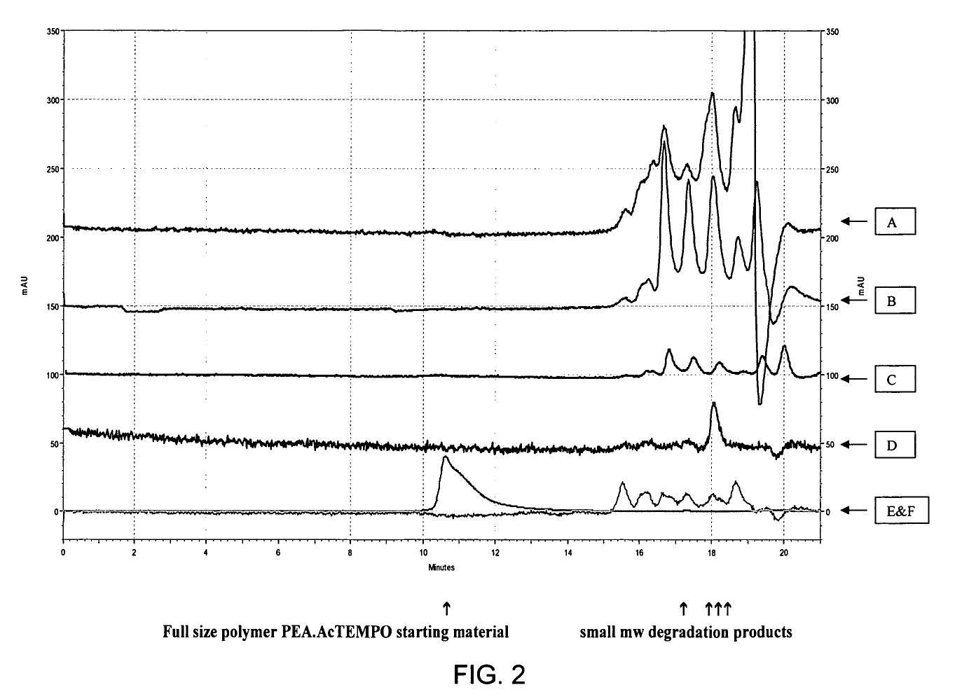

The GPC traces (THF, PS, PLGel C+E column) for PEA.Ac.Bz are shown in FIG. 1 and for PEA.Ac.TEMPO in FIG. 2. The traces confirm that macrophages can degrade PEA, but it was not determined whether the major mechanism was via secretion of enzymes or via uptake of polymer with further degradation occurring intracellularly and degradation products being subsequently secreted back into the media.

Use of dansylated-PEA enabled visualization of the polymer associated with the macrophages. Cells from some wells were trypsinized for removal from the surface of the wells and replated into tissue culture polystyrene wells. The macrophages retained fluorescent material, indicating that the PEA film had been degraded and taken into the cells.

### EXAMPLE 7

An important feature of the invention PEA polymers is their ability to promote a natural healing response. To gain insight into this process, in the following series of examples, PEA was compared to non-degradable and other biodegradable polymers in a series of in vitro assays to examine blood and cellular responses to the polymers that are important for healing of adjacent tissue after placement of an invention adhesion barrier.

Tissue compatibility was measured by exposing human peripheral blood monocytes to PEA, PEA-TEMPO, 50:50 poly(D,L-lactide-co-glycolide) (PLGA), poly(n-butyl methacrylate) (PBMA) and tissue culture-treated polystyrene (TCPS).

Human peripheral blood monocytes were isolated by density centrifugation and magnetic separation (Miltenyi). PLGAs of 34,000 and 73,000 Da average molecular weight were purchased from Boehringer-Ingelheim. PBMA was purchased from Polysciences. TCPS plates (Falcon) with or without fibronectin, fibrinogen, heparin, or gelatin (Sigma) coatings were used as controls.

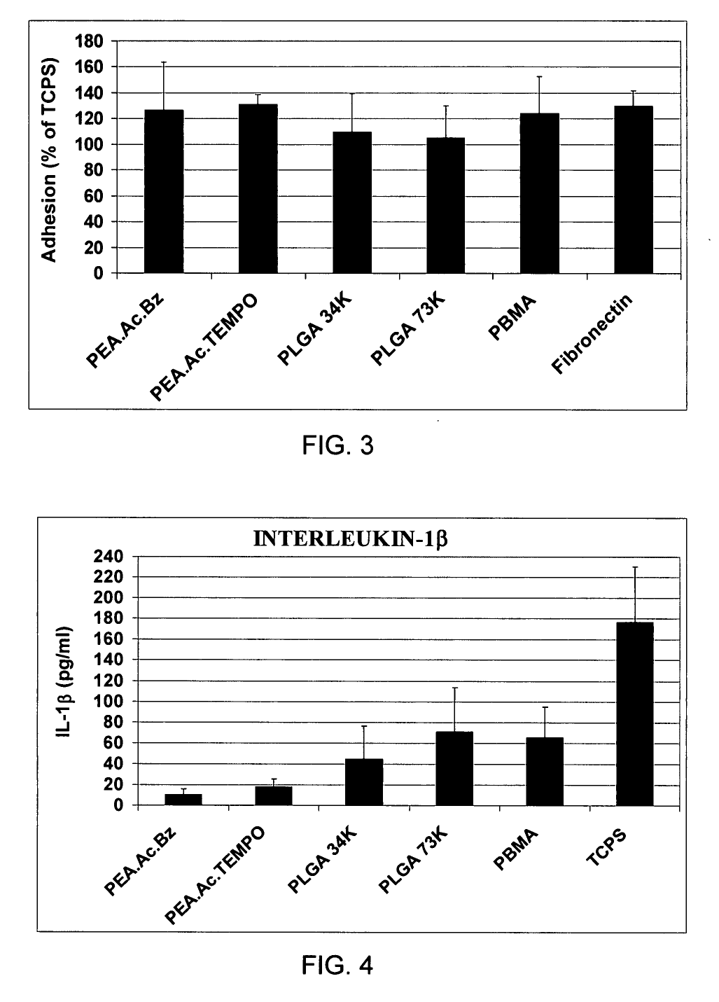

Human monocytes were seeded at 1.6×105/cm2 into wells containing polymers cast on cover-slips. Cells were incubated for 24 hours, and adhesion was measured by quantifying cellular ATP levels (ViaLight Kit, Cambrex). Equivalent numbers of monocytes adhered to each polymer (n=6) (FIG. 3).

Phenotypic progression of monocytes-to-macrophages and contact-induced fusion to form multinucleated cells proceeded at similar rates (FIG. 3) over three weeks of culture. PEA surfaces supported adhesion and differentiation of human monocytes, but, qualitatively, PEA surfaces do not appear to induce a hyper-activated state as judged by microscopic visualization of morphology and differentiation/fusion rates over a 20 day period. Freshly isolated monocytes are approximately 10 μm in diameter and non-granular in appearance. After adherence to PEA, the monocytes flattened on the surface and assumed either a motile (triangular-shaped) or non-motile (circular) phenotype that is common for this heterogeneous population. Over the following 5-7 days, the monocytes differentiated into macrophages, as judged by an increase in cell size to greater than 20 μm in diameter and increased granularity. The macrophages remained viable in culture for the full 20 day culture period, and there was a low degree of fusion of macrophages to form multinucleated cells.

### EXAMPLE 7

Secretion of pro-inflammatory and anti-inflammatory mediators by monocytes and macrophages were measured by ELISA (R&D Systems) after 24 hours of incubation of the monocytes and macrophages on the polymers.

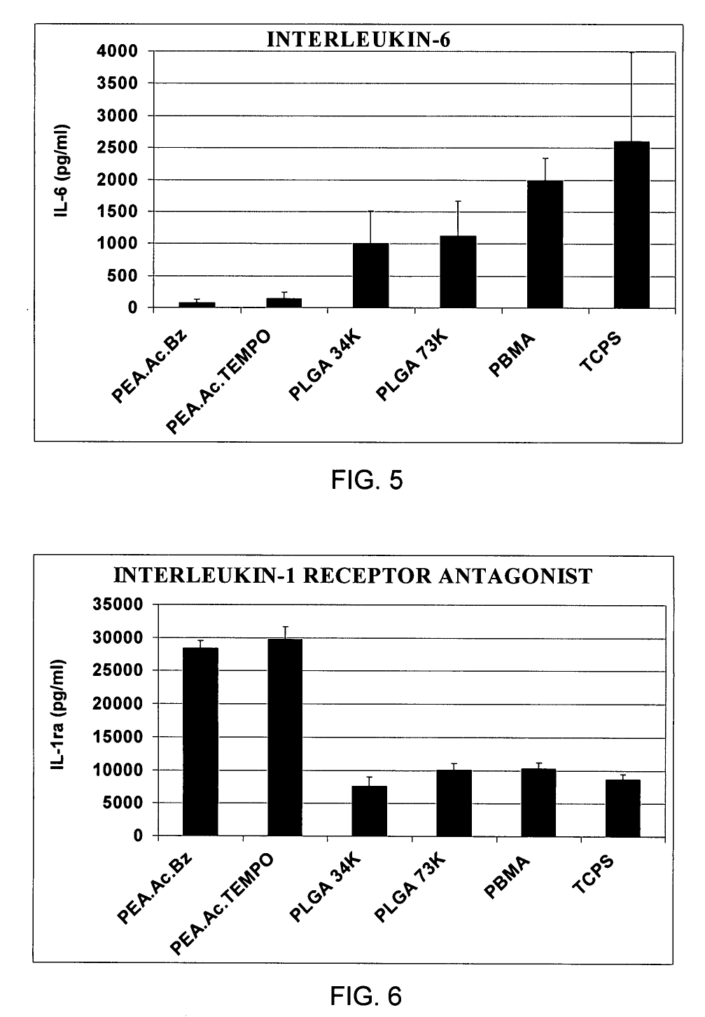

Interleukin-6 is a pleiotropic pro-inflammatory cytokine that can increase macrophage cytotoxic activities. Monocytes secreted over 5-fold less IL-6 (FIG. 5) when on PEAs than on the other polymers (representative experiment of n=4).

### EXAMPLE 8

Interleukin-1β is a potent pro-inflammatory cytokine that can increase the surface thrombogenicity of the endothelium. After 24 hours, monocytes incubated on the PEAs secreted less IL-1β than those incubated on PLGA 73K or on PBMA (FIG. 4) (representative experiment of n=4).

As shown in FIGS. 5 and 6, PEA polymers induce the lowest inflammatory response and also induce the highest anti-inflammatory response, which limits runaway inflammation

These in vitro assessments of the tissue compatibility and inflammatory response to PEA biodegradable, amino acid-based polymers suggest that implantable adhesion barriers based on such polymers would afford a more natural healing response and be less prone to cause inflammation than the other polymers tested by attenuating the pro-inflammatory reaction to the polymer and promoting re-endothelialization. In addition, the suppression of platelet activation strongly indicates that the PEA polymers are highly hemocompatible. Taken together, these results suggest that PEA and PEA-TEMPO are superior biodegradable polymers for use in adhesion barriers.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A composition comprising at least one biodegradable polymer dissolved in a biocompatible liquid solvent, wherein the polymer comprises at least one of a poly(ester amide) (PEA) having a chemical formula described by structural formula (I), 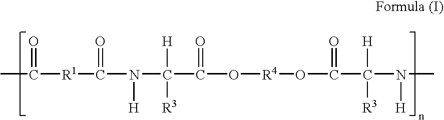
wherein n ranges from about 5 to about 150; R¹ is independently selected from the group consisting of (C₂-C₂₀) alkylene, (C₂-C₂₀) alkenylene, α,ω-bis(4-carboxyphenoxy) (C₁-C₈) alkane, residues of saturated and unsaturated adhesion preventing di-acids, residues of α,ω-alkylene dicarboxylates of formula (III), and combinations thereof; wherein R⁵ and R⁷ in Formula (III) are each independently selected from (C₂-C₁₂) alkylene or (C₂-C₁₂) alkenylene; the R³s in individual n monomers are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing di-acids, and combinations thereof; 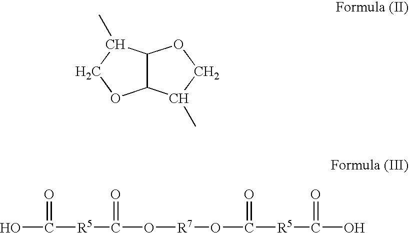
or a PEA having a chemical formula described by structural formula (IV), 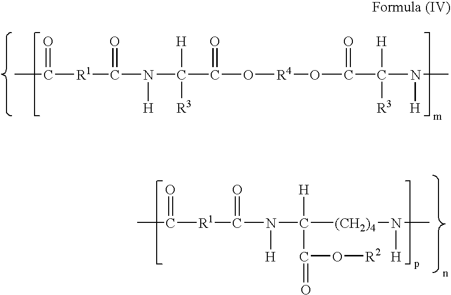
wherein n ranges from about 5 to about 150, m ranges about 0.1 to 0.9: p ranges from about 0.9 to 0.1; wherein R¹ is independently selected from the group consisting of (C₂-C₂₀) alkylene, (C₂-C₂₀) alkenylene, α,ω-bis(4-carboxyphenoxy) (C₁-C₈) alkane, and residues of saturated and unsaturated adhesion preventing di-acids, residues of α,ω-alkylene dicarboxylates of formula (III), and combinations thereof; wherein R⁵ and R⁷ in Formula (III) are each independently selected from (C₂-C₁₂) alkylene or (C₂-C₁₂) alkenylene; each R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R³s in individual m monomers are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols and combinations thereof;
or a poly(ester urethane) (PEUR) having a chemical formula described by structural formula (V), 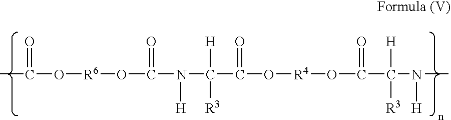
and wherein n ranges from about 5 to about 150; wherein the R³s within an individual n monomer are independently selected from the group consisting of hydrogen, (C₁-C₆) alkyl, (C₂-C₆) alkenyl, (C₆-C₁₀) aryl(C₁-C₆) alkyl and —(CH₂)₂S(CH₃); R⁴ and R⁶ are each selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols, and combinations thereof;
or a PEUR having a chemical structure described by general structural formula (VI), 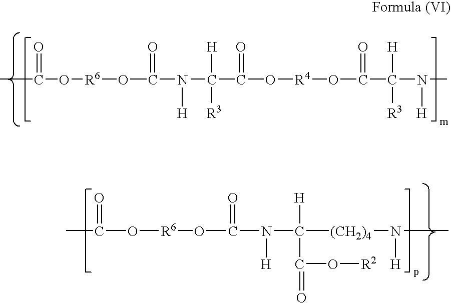
wherein n ranges from about 5 to about 150, m ranges about 0.1 to about 0.9: p ranges from about 0.9 to about 0.1; R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R³s within an individual m monomer are independently selected from the group consisting of hydrogen, (C₁-C₆) alkyl, (C₂-C₆) alkenyl, (C₆-C₁₀) aryl (C₁-C₆) alkyl and —(CH₂)₂S(CH₃); R⁴ and R⁶ are each independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), residues of saturated and unsaturated adhesion preventing diols, and combinations thereof;
or a poly(ester urea) (PEU) having a chemical formula described by structural formula (VII), 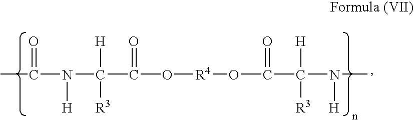
wherein n is about 10 to about 150; the R³s within an individual n monomer are independently selected from the group consisting of hydrogen, (C₁-C₆) alkyl, (C₂-C₆) alkenyl, (C₆-C₁₀) aryl (C₁-C₆)alkyl and —(CH₂)₂S(CH₃); R⁴ is independently selected from the group consisting of (C₂-C₂₀) alkylene, (C₂-C₂₀) alkenylene, (C₂-C₈) alkyloxy (C₂-C₂₀) alkylene, residues of a saturated and unsaturated adhesion preventing diols, bicyclic-fragments of a 1,4:3,6-dianhydrohexitol of structural formula (II) and combinations thereof;
or a PEU having a chemical formula described by structural formula (VIII); 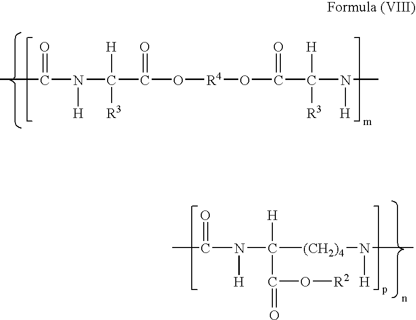
wherein m is about 0.1 to about 1.0; p is about 0.9 to about 0.1; n is about 10 to about 150; each R² is independently selected from the group consisting of hydrogen, (C₁-C₁₂) alkyl, (C₂-C₈) alkyloxy (C₂-C₂₀) alkyl, (C₆-C₁₀) aryl and a protecting group; and the R³s within an individual m monomer are independently selected from the group consisting of hydrogen, (C₁-C₆) alkyl, (C₂-C₆) alkenyl, (C₆-C₁₀) aryl (C₁-C₆)alkyl and —(CH₂)₂S(CH₃); R⁴ is independently selected from the group consisting of (C₂-C₂₀) alkylene, (C₂-C₂₀) alkenylene, (C₂-C₈) alkyloxy (C₂-C₂₀) alkylene, residues of saturated and unsaturated adhesion preventing diols; bicyclic-fragments of a 1,4:3,6-dianhydrohexitol of structural formula (II), and combinations thereof,
wherein the composition forms a biodegradable adhesion barrier when applied to a tissue surface.

2. The composition of claim 1, wherein the biocompatible solvent comprises ethanol.

3. The composition of claim 1, wherein the composition has a sprayable viscosity.

4. The composition of claim 1, wherein the composition forms a first thin tissue adherent layer when sprayed or painted onto a tissue surface and allowed to dry.

5. The composition of claim 4, wherein the polymer of the first barrier layer has a weight average molecular weight in the range from about 5,000 Da to about 25,000 Da.

6. The composition of claim 5, wherein the composition further comprises a second thin barrier layer of the polymer that forms a substantially non-tissue adherent layer when sprayed or painted onto the first layer and allowed to dry.

7. The composition of claim 6, wherein the polymer of the second non-tissue adherent layer has a weight average molecular weight in the range from about 85,000 Da to about 300,000 Da.

8. The composition of claim 1, further comprising at least one adhesion preventing bioactive agent dispersed in the polymer.

9. The composition of claim 1, wherein a residue of a di-acid or diol adhesion preventing bioactive agent is contained in the backbone of the polymer.

10. The composition of claim 1, wherein the composition is formulated to biodegrade over a period of from about 3 days to about 6 months.

11. The composition of claim 1, wherein the composition is used to fabricate a first preformed adhesive solid sheet or layer.

12. The composition of claim 11, wherein the solid sheet or layer is porous.

13. The composition of claim 1, wherein the composition is used to fabricate first and second solid layers, each comprising a different one of the polymers such that the first layer overlies the second layer and has a substantially higher adherence to flesh than the second layer.

14. The composition of claim 13, wherein at least one of the first layer and the second layer further comprises an adhesion preventing bioactive agent dispersed in the polymer.

15. The composition of claim 13, wherein each of the first layer and second layer each has a thickness of about 0.1 mm to about 2.5 mm.

16. The composition of claim 13, wherein the polymer of the first layer has a weight average molecular weight in the range from about 5,000 to about 25,000 and the polymer of the second layer has a weight average molecular weight in the range from about 85,000 to about 300,000.

17. The composition of claim 1, wherein the polymer has the chemical formula described by structural formula (I), (V) or (VII) and R³s in at least one monomer n is CH₂Ph.

18. The composition of claim 1, wherein the 1,4:3,6-dianhydrohexitol of structural formula (II) is derived from D-glucitol, D-mannitol, or L-iditol.

19. The composition of claim 1, wherein the composition biodegrades over a period of about 3 days to about 6 months.

20. A method of applying an adhesion barrier to a tissue surface, said method comprising:
applying the composition of claim 1 to 19 to the tissue surface so as to adhere the composition to the tissue surface.

21. The method of claim 20, wherein the composition is applied by spraying or painting the tissue surface with the composition and allowing the composition to dry.

22. The method of claim 20, wherein the composition is prefabricated to form at least one solid sheet by spraying or painting the composition onto a solid surface.

23. A method of preventing post-surgical adhesions in a subject undergoing open surgery comprising
applying a composition of claim 1 to at least one tissue surface at a surgical opening so as to form a tissue-adhesive adhesion barrier between opposing tissue surfaces or a tissue-organ surface;
closing the surgical opening while maintaining the composition in place for a sufficient time to prevent in-growth of scar tissue and the formation or reformation of adhesions immediately adjacent to the composition while injured surfaces heal.

24. The method of claim 23, wherein the composition further comprises at least one adhesion preventing bioactive agent dispersed in the polymer.

25. The method of claim 23, wherein the sufficient time is from about three days to about 6 months.
